(19) United States
(12) Patent Application Publication
(10) Pub. No.: US 2009/0105346 A1
(43) Pub. Date: Apr. 23, 2009

(54) Novel crystalline forms of armodafinil and preparation thereof

(76) Inventor: Alexandr Jegorov, Dobra Voda (CZ)

Correspondence Address: KENYON & KENYON LLP, ONE BROADWAY, NEW YORK, NY, 10004, US

(21) Appl. No.: 12/287,011

(22) Filed: Oct. 2, 2008

Related U.S. Application Data
(60) Provisional application No. 60/977,000, filed on Oct. 2, 2007.

(51) Int. Cl. A61K 31/165; C07C 233/11

(52) U.S. Cl. 514/618; 564/162

(57) ABSTRACT

The invention encompasses crystalline forms of armodafinil, processes for preparing the crystalline forms of armodafinil, pharmaceutical formulation thereof, and method of treating excessive sleepiness using the formulations of the invention.

### CROSS-REFERENCE TO RELATED APPLICATIONS

This application claims the benefit of U.S. Provisional Application No. 60/977,000, filed Oct. 2, 2007, hereby incorporated by reference.

### FIELD OF THE INVENTION

The invention encompasses crystalline forms of armodafinil, processes for preparing the crystalline forms of armodafinil, pharmaceutical formulation thereof, and method of treating excessive sleepiness using the formulations of the invention.

### BACKGROUND OF THE INVENTION

Modafinil is currently marketed by Cephalon, Inc. under the trade name Provigil® as a racemic mixture of its R and S enantiomers. Provigil® is indicated for the treatment of excessive sleepiness associated with narcolepsy, shift work sleep disorder (SWSD), and obstructive sleep apnea/hypopnea syndrome (OSA/HS).

Studies have shown that while both enantiomers of modafinil are pharmacologically active, the S enantiomer is eliminated from the body three times faster than the R enantiomer. Prisinzano et al., Tetrahedron: Asymmetry, vol. 5 1053-1058 (2004). It is, therefore, preferable to develop pharmaceutical compositions of the R enantiomer of modafinil, as opposed to its racemic mixture.

The R enantiomer of modafinil is known as armodafinil and has the chemical name 2-[(R)-(diphenylmethyl)sulfinyl]acetamide. The molecular weight of armodafinil is 273.34 and it has the following chemical structure:

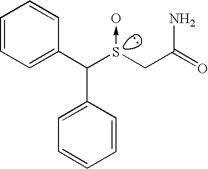

Armodafinil is commercially available as Nuvigil™.

Armodafinil and process for its preparation were first disclosed in U.S. Pat. No. 4,927,855 (“'855 patent”) and EP Publication No. 0233106, both of which were originally assigned to Laboratoire L. Lafon. The '855 patent purportedly describes the synthesis of armodafinil as shown in the following general scheme:

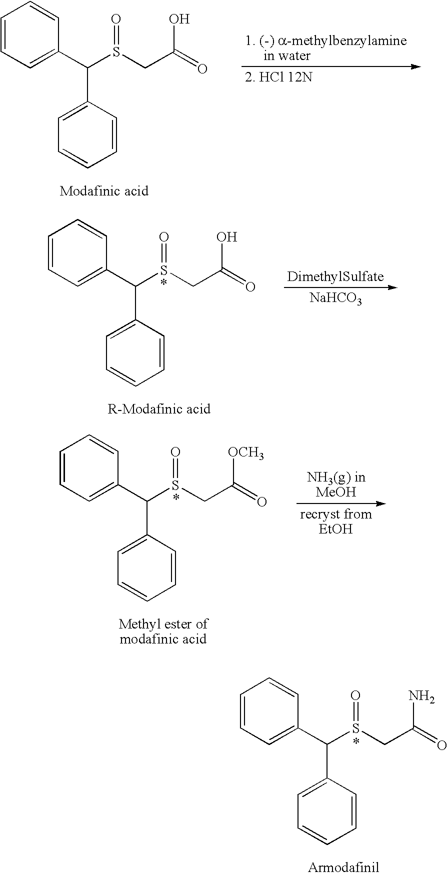

See '855 patent, col. 2, 11. 16-53.

Armodafinil can exist in several crystalline forms, some of which are disclosed in WO 2005/023198 (“WO '198”), WO 2005/077894 (“WO '894”), and WO 2004/060858 (“WO '858”).

WO '858 purportedly discloses armodafinil Forms I-IV and an amorphous form, as well as solvates of armodafinil. WO '894 purportedly discloses armodafinil Forms III-V, as well as solvates of armodafinil, and WO '198 purportedly discloses solvates of armodafinil.

The occurrence of different crystal forms (polymorphism) is a property of some molecules and molecular complexes. A single molecule, like armodafinil, may give rise to a variety of solids having distinct physical properties such as melting point, X-ray diffraction pattern, infrared absorption fingerprint, and NMR spectrum. The differences in the physical properties of polymorphs result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. One of the most important physical properties of polymorphs in pharmaceutical is their solubility in aqueous solution. Polymorphs exhibit different solubilities in aqueous solution which may affect the dissolution rate of a drug and consequently affect its bioavailability in the body. Pharmaceutical drugs are often administered orally as a crystalline solid and dissolution rates depend on the exact crystal form of a polymorph. For example, polymorphs in a more rapidly dissolving form are likely to be more effective over a more slowly dissolving form for drugs that are absorbed rapidly. On the other hand, drugs that are slowly absorbed may also be unstable in the gastrointestinal tract and therefore benefit from a slow dissolution rate as not to increase the drug concentration in this detrimental environment. Therefore, polymorphism is important in the development of pharmaceutical ingredients.

The discovery of new forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Thus, there remains a need in the art for additional forms of armodafinil and/or processes for their preparation.

### SUMMARY OF THE INVENTION

One embodiment of the invention encompasses a crystalline form of armodafinil characterized by a powder XRD pattern having peaks at about 12.4, 14.3, 17.2, 18.1, 21.6, and 23.2±0.2 degrees 2-theta, herein defined as armodafinil form SJ1.

Another embodiment of the invention encompasses a process for preparing armodafinil form SJ1 comprising dissolving armodafinil in xylene; heating; cooling to obtain a precipitate; and isolating the precipitate.

Another embodiment of the invention encompasses a crystalline form of armodafinil characterized by a powder XRD pattern having peaks at about 5.2, 10.5, 13.8, 18.5, and 19.0±0.2 degrees 2-theta, herein defined as armodafinil form SJ6.

Another embodiment of the invention encompasses a process for preparing armodafinil form SJ6 comprising combining armodafinil form I with dimethyl sulfoxide to obtain a suspension; maintaining the suspension to obtain a precipitate; and isolating the precipitate.

Another embodiment of the invention encompasses a pharmaceutical formulation comprising a therapeutically effective amount of at least one of crystalline form SJ1 or SJ6 of armodafinil and at least one pharmaceutically acceptable excipient.

Another embodiment of the invention encompasses a process for preparing a pharmaceutical formulation of armodafinil comprising combining at least one of crystalline forms SJ1 or SJ6 of armodafinil and at least one pharmaceutically acceptable excipient.

Another embodiment of the invention encompasses the use of a pharmaceutical formulation comprising a therapeutically effective amount of at least one of crystalline form SJ1 or SJ6 of armodafinil and at least one pharmaceutically acceptable excipient in the manufacture of a medicament for treatment of excessive sleepiness associated with narcolepsy, shift work sleep disorder (“SWSD”), and obstructive sleep apnea/hypopnea syndrome (“OSA/HS”)

### BRIEF DESCRIPTION OF THE FIGURES

FIG. 1 illustrates the powder XRD pattern of armodafinil Form SJ1.

FIG. 2 illustrates the powder XRD pattern of armodafinil Form SJ6.

### DETAILED DESCRIPTION OF THE INVENTION

The invention encompasses crystalline forms of armodafinil and processes for making these crystalline forms of armodafinil. Each solid form possesses properties that are unique and useful to the pharmaceutical formulator during formulation. These and other properties may be advantageous to the process chemist when designing scale-up synthesis, purification, and/or storage conditions of armodafinil. In addition, the processes described herein are useful in the production of crystalline forms of armodafinil in laboratory and commercial scale operations.

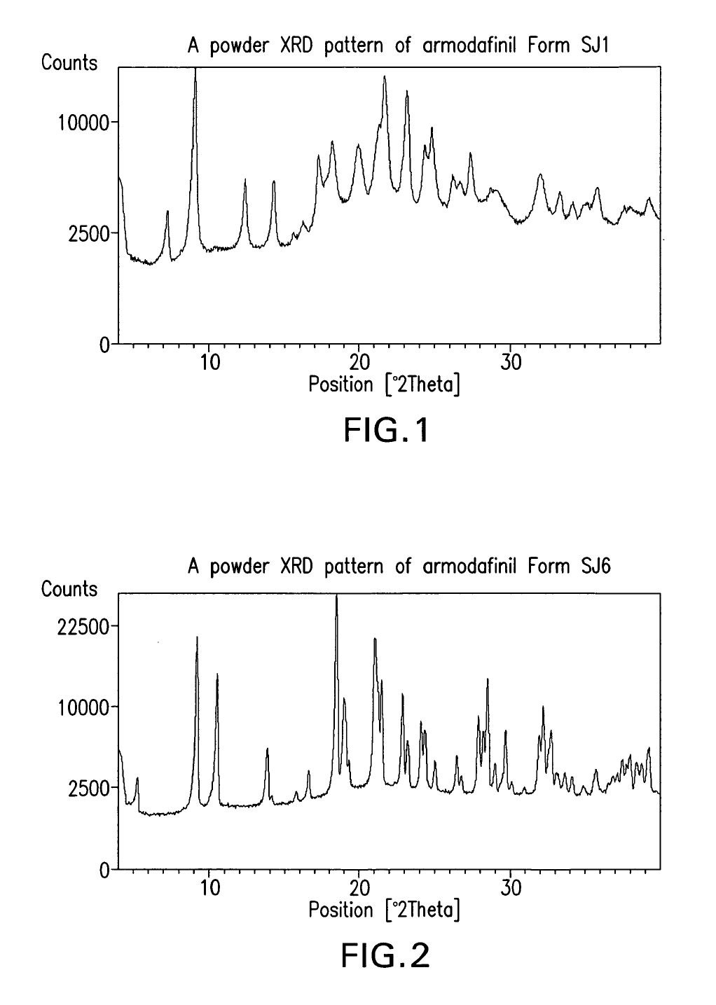

One embodiment of the invention encompasses a crystalline form of armodafinil characterized by a powder XRD pattern having peaks at about 12.4, 14.3, 17.2, 18.1, 21.6, and 23.2±0.2 degrees 2-theta, herein defined as armodafinil form SJ1. Armodafinil form SJ1 may be further characterized by a powder XRD pattern having additional peaks at about 7.2, 9.1, and 20.1±0.2 degrees 2-theta or a powder XRD pattern as substantially depicted in FIG. 1.

The invention also encompasses a process for preparing armodafinil form SJ1 by crystallizing armodafinil from xylene. The process comprises dissolving armodafinil in xylene; heating; cooling to obtain a precipitate; and isolating the precipitate.

Armodafinil may be prepared according to U.S. Pat. No. 4,927,855, hereby incorporated by reference. Optionally, armodafinil form I can be used as the starting armodafinil. Armodafinil form I may be prepared according to WO 2004/060858, hereby incorporated by reference.

Typically, xylene is present in a form of one of its isomers or as a mixture of xylene isomers. For example, xylene is selected from the group consisting of o-xylene, m-xylene, p-xylene, and mixtures thereof Preferably, p-xylene or m-xylene is used. Preferably, xylene is present in an amount of about 20 ml to 60 ml per gram of armodafinil. More preferably, xylene is present in an amount of about 30 ml to about 50 ml per gram of armodafinil.

Typically, armodafinil is dissolved in xylene while heating. Preferably, the heating is at a temperature of about 50° C. to about 140° C. More preferably, the heating is at a temperature of about 65° C. to 115° C. Preferably, the heating is for about 10 to 30 minutes. Most preferably, the heating is a temperature of about 75° C. to 115° C. for about 15 minutes.

Typically, when the solvent is p-xylene, the cooling step is performed at a temperature of about 30° C. to about 13° C., and preferably, at a temperature of about 25° C. to about 15° C. More preferably, the cooling step is done at a temperature of about 20° C. Typically, when the solvent is m-xylene or a mixture of xylenes, the cooling step is performed at a temperature of about 30° C. to about 0° C., preferably, at a temperature of about 25° C. to about 15° C. More preferably, the cooling step is done at a temperature of about 20° C.

Preferably, the cooling step is done for about 1 hour to about 20 hours, and more preferably, about 2 hours to about 12 hours. Most preferably, it is done for about overnight. As used herein, the term “overnight” refers to a period of time of about 12 hours.

The isolation can be achieved using any method known in the art, for example, by filtration. Generally, the crystals of armodafinil Form SJ1 are isolated by filtration. Preferably, after the filtration step, the crystals are washed. Preferably the washing is with C₆-C₈ aliphatic and aromatic hydrocarbons or mixtures thereof. More preferably, the washing is with a solvent selected from the group consisting of: hexane, heptane, cyclohexane and petroleum ether. Most preferably, the washing is with n-hexane. Preferably, after the washing, the crystals are air dried. Optionally, the drying can be done under vacuum.

As described herein, when referring to drying crystalline armodafinil under vacuum, the term “vacuum” refers to vacuum drying under a pressure of about 100 mbar to about 0.1 mbar, preferably, under a pressure of about 10 mbar to about 1 mbar.

Another embodiment of the invention encompasses a crystalline form of armodafinil characterized by a powder XRD pattern having peaks at about 5.2, 10.5, 13.8, 18.5 and 19.0±0.2 degrees 2-theta, herein defined as armodafinil form SJ6. Armodafinil form SJ6 may be further characterized by a powder XRD pattern having additional peaks at about 9.2, 21.0 and 22.9±0.2 degrees 2-theta or a powder XRD pattern as substantially depicted in FIG. 2.

The invention further encompasses a process for preparing armodafinil form SJ6 by suspending armodafinil form I in dimethyl sulfoxide (“DMSO”). The process comprising combining armodafinil form I with DMSO to obtain a suspension; maintaining the suspension to obtain a precipitate; and isolating the precipitate.

Preferably, DMSO is present in an amount of about 0.2 ml to about 0.7 ml per gram of armodafinil. More preferably, DMSO is present in amount of about 0.3 ml to about 0.5 ml per gram of armodafinil.

Preferably, the suspension is maintained at a temperature of about 15° C. to about 40° C., and more preferably, at a temperature of about 19° C. to about 40° C. Most preferably, the suspension is maintained at a temperature of 20° C. Preferably, the suspension is maintained for about 1 hour to about 20 hours, and more preferably, for about 2 hours to about 12 hours. Most preferably, it is maintained for about overnight.

The isolation can be achieved using any method known in the art, for example, by filtration.

Another embodiment of the invention encompasses a pharmaceutical formulation comprising a therapeutically effective amount of at least one of crystalline form SJ1 or SJ6 of armodafinil and at least one pharmaceutically acceptable excipient.

The present invention also encompasses a process for preparing such pharmaceutical formulations comprising combining at least one of crystalline forms SJ1 or SJ6 of armodafinil and at least one pharmaceutically acceptable carrier. These pharmaceutical formulations can be used for treatment of excessive sleepiness.

Therapeutically effective amounts of armodafinil include, but are not limited to, 50 mg, 150 mg, and 250 mg per dosage forms of armodafinil.

Pharmaceutically acceptable excipients may include excipients commonly used in pharmaceutical formulations. Pharmaceutically acceptable excipients used in the formulation include, but are not limited to, diluents, binders, disintegrants, lubricants, flavorings, sweeteners, or preservatives.

Diluents used in the formulation include diluents commonly used in pharmaceutical formulations. For example, diluents include, but are not limited to, cellulose-derived materials, such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents, such as calcium carbonate and calcium diphosphate; waxes; sugars; sugar alcohols, such as mannitol and sorbitol; acrylate polymers and copolymers; pectin; dextrin; or gelatin.

Binders used in the formulation include binders commonly used in pharmaceutical formulations. For example, binders include, but are not limited to, acacia gum, pregelatinized starch, sodium alginate, or glucose.

Disintegrants used in the formulation include disintegrants commonly used in pharmaceutical formulations. For example, disintegrants include, but are not limited to, sodium starch glycolate, crospovidone, or low-substituted hydroxypropyl cellulose.

Lubricants used in the formulation include lubricants commonly used in pharmaceutical formulations. For example, lubricants include, but are not limited to magnesium stearate, calcium stearate, or sodium stearyl fumarate.

Flavoring agents and flavor enhancers make the dosage form more palatable to the patient. Common flavoring agents and flavor enhancers for pharmaceutical products that can be included in the composition of the invention include maltol, vanillin, ethyl vanillin, menthol, citric acid, fumaric acid, ethyl maltol, and tartaric acid.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxy toluene, butylated hydroxyanisole, and ethylenediamine tetraacetic acid can be added at levels safe for ingestion to improve storage stability.

The pharmaceutical formulations of the invention may be provided in dosage forms for oral, parenteral (including subcutaneous, intramuscular, and intravenous), inhalant, or ophthalmic administration. Although the most suitable route in any given case will depend on the nature and severity of the condition being treated, the most preferred route of the invention is oral. Dosages may be conveniently presented in unit dosage form and prepared by any of the methods well-known in the art of pharmacy.

Dosage forms include solid dosage forms, such as tablets, powders, capsules, suppositories, sachets, troches, and lozenges, as well as liquid suspensions and elixirs.

Capsule dosages will contain the solid composition within a capsule which may be made of gelatin or other conventional encapsulating material.

Tablets and powders may be coated, for example, with an enteric coating. The enteric-coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Another embodiment of the invention encompasses methods of treating patients suffering from excessive sleepiness associated with narcolepsy, shift work sleep disorder (“SWSD”), and obstructive sleep apnea/hypopnea syndrome (“OSA/HS”) comprising administering to a patient in need thereof a therapeutically effective amount of a pharmaceutical formulation comprising at least one crystalline form SJ1 or SJ6 of armodafinil and at least one pharmaceutically acceptable excipient.

Another embodiment of the invention encompasses the use of a pharmaceutical formulation comprising a therapeutically effective amount of at least one of crystalline form SJ1 or SJ6 of armodafinil and at least one pharmaceutically acceptable excipient in the manufacture of a medicament for treatment of excessive sleepiness associated with narcolepsy, shift work sleep disorder (“SWSD”), and obstructive sleep apnea/hypopnea syndrome (“OSA/HS”)

Having described the invention with reference to certain preferred embodiments, other embodiments will become apparent to one skilled in the art from consideration of the specification. The invention is further defined by reference to the following examples describing in detail the preparation of armodafinil crystalline forms of the invention. It will be apparent to those skilled in the art that many modifications, both to materials and methods, may be practiced without departing from the scope of the invention.

### EXAMPLES

### PXRD

X-ray powder diffraction data were obtained by the following method. A powder X-ray diffractometer model Philips X'pert Pro was employed with CuK_{α} radiation of 1.5418 Å. An X'Celerator detector with active length (2 theta)=2.122 degrees was used and the laboratory temperature of 22-25° C. was used. The scanning parameters were: range: 4-40 degrees two-theta; scan mode: continuous scan; step size: 0.0167 degrees; and scan step time was 30.8 seconds. All peak positions are within ±0.2 degrees two theta.

### Example 1

Preparation of Armodafinil Form SJ1 from m-Xylene

Armodafinil Form I (300 mg) was added to 10 ml m-xylene and heated at 75° C. for about 15 minutes forming a solution. The solution was allowed to cooled to 20° C. within about 20 minutes and then it was allowed to stand overnight which resulted in the formation of crystals. The crystals were subsequently filtered, washed with 10 ml n-hexane and air dried for about 1 hour (Yield=242 mg). The crystals were analyzed by powder X-ray diffractometer and identified as armodafinil Form SJ1.

### Example 2

Preparation of Armodafinil Form SJ1 from p-Xylene

Armodafinil Form I (300 mg) was added to 15 ml p-xylene and heated at 100° C. for about 15 minutes forming a solution. The solution was allowed to cooled to 20° C. within about 20 minutes and then it was allowed to stand overnight which resulted in the formation of crystals. The crystals were subsequently filtered, washed with 10 ml n-hexane and dried on air for about 1 hour (Yield=181 mg). The crystals were analyzed by powder X-ray diffractometer and identified as armodafinil Form SJ1.

### Example 3

### Preparation of Armodafinil Form SJ6

Armodafinil Form I (300 mg) was mixed with 150 μl dimethyl sulfoxide and allowed to stand overnight at 20° C. Within this time, the original suspension solidified and crystals were formed. The crystals were analyzed by powder X-ray diffractometer and identified as armodafinil Form SJ6.

We claim:

1. A crystalline form of armodafinil characterized by a powder XRD pattern having peaks at about 12.4, 14.3, 17.2, 18.1, 21.6, and 23.2±0.2 degrees 2-theta.

2. The crystalline form according to claim 1 further characterized by a powder XRD pattern having peaks at about 7.2, 9.1, and 20.1±0.2 degrees 2-theta.

3. The crystalline form according to claim 1 further characterized by a PXRD pattern substantially depicted in FIG. 1.

4. A process for preparing armodafinil form SJ1 comprising dissolving armodafinil in xylene; heating; cooling to obtain a precipitate; and isolating the precipitate.

5. The process according claim 4, wherein the xylene is p-xylene or m-xylene.

6. The process according to claim 4, wherein xylene is present in an amount of about 20 ml to 60 ml per gram of armodafinil.

7. The process according to claim 4, wherein dissolving in xylene is done while heating.

8. The process according to claim 4, wherein the heating is at a temperature of about 50° C. to about 140° C.

9. The process according to claim 4, wherein the cooling is at a temperature of about 30° C. to about 0° C.

10. The process according to claim 4, wherein the cooling step is carried out for about 2 hours to about 12 hours.

11. A crystalline form of armodafinil characterized by a powder XRD pattern having peaks at about 5.2, 10.5, 13.8, 18.5, and 19.0±0.2 degrees 2-theta.

12. The crystalline form according to claim 11 further characterized by a powder XRD pattern having peaks at about 9.2, 21.0, and 22.9±0.2 degrees 2-theta.

13. The crystalline form according to claim 11 further characterized by a PXRD pattern substantially depicted in FIG. 2.

14. A process for preparing armodafinil form SJ6 comprising combining armodafinil form I with dimethyl sulfoxide to obtain a suspension; maintaining the suspension to obtain a precipitate; and isolating the precipitate.

15. The process according to claim 14, wherein dimethyl sulfoxide is present in an amount of about 0.2 ml to about 0.7 ml per gram of armodafinil.

16. The process according to claim 14, wherein the suspension is maintained at a temperature of about 15° C. to about 40° C.

17. The process according to claim 14, the suspension is maintained for about 2 hours to about 12 hours.

18. A pharmaceutical formulation comprising a therapeutically effective amount of at least one of crystalline form SJ1 or SJ6 of armodafinil and at least one pharmaceutically acceptable excipient.

19. A process for preparing a pharmaceutical formulation of armodafinil comprising combining at least one of crystalline forms SJ1 or SJ6 of armodafinil and at least one pharmaceutically acceptable excipient.

20. A method of treating patients suffering from excessive sleepiness comprising administering to a patient in need thereof a therapeutically effective amount of a pharmaceutical formulation comprising at least one crystalline form SJ1 or SJ6 of armodafinil and at least one pharmaceutically acceptable excipient.
